(19) United States
(12) Patent Application Publication
(10) Pub. No.: US 2007/0123793 A1
(43) Pub. Date: May 31, 2007

(54) Apparatus for Evaluating a Patient's Laryngeal Cough Reflex and Associated Methods

(75) Inventors: W. Robert Addington, Melbourne Beach, FL (US); Stuart Miller, Melbourne Beach, FL (US); Robert Stephens, Kansas City, MO (US)

Correspondence Address: ALLEN, DYER, DOPPELT, MILBRATH & GILCHRIST P.A., 1401 CITRUS CENTER 255 SOUTH ORANGE AVENUE, P.O. BOX 3791, ORLANDO, FL, 32802-3791, US

(73) Assignee: Pneumoflex Systems, LLC., Melbourne Beach, FL (US)

(21) Appl. No.: 11/626,500

(22) Filed: Jan. 24, 2007

Related U.S. Application Data
(62) Division of application No. 10/783,442, filed on Feb. 20, 2004.
(60) Provisional application No. 60/448,915, filed on Feb. 20, 2003.

(51) Int. Cl. A61B 5/04

(52) U.S. Cl. 600/546

(57) ABSTRACT

An apparatus and method for evaluating a patient's laryngeal cough reflex function includes a nebulizer capable of being actuated to atomize a cough-inducing substance, a switch associated with the nebulizer, the switch responsive to actuation of the nebulizer, and a connection between the switch and an EMG machine to thereby activate the EMG machine responsive to the switch. Elapsed time between switch actuation and electrical activity sensed in a muscle which contracts to produce a cough is indicative of status of the patient's laryngeal cough reflex.

### RELATED APPLICATION

This application claims priority from co-pending provisional application Ser. No. 60/448,915, which was filed on Feb. 20, 2003, and which is incorporated herein by reference in its entirety.

### FIELD OF THE INVENTION

The present invention relates to medical devices and, more particularly, to an apparatus for evaluating a patient's laryngeal cough reflex and to its associated methods.

### BACKGROUND OF THE INVENTION

A patient's ability to produce a cough has been used as an indicator of return of the patient's laryngeal cough reflex following an event which suppresses that reflex. For example, post operative patients who are emerging from the effects of intubation for administration of breathing assistance and/or anesthetics during surgery must be evaluated for return of the laryngeal reflex, as these patients will remain susceptible to aspiration of foreign matter into the respiratory airways while the laryngeal cough reflex remains suppressed.

In addition, various medical conditions may give rise to complete or partial suppression of the normal laryngeal cough reflex. Those skilled in the art will understand that the laryngeal cough reflex includes the closing of the larynx, i.e., glottal closure, to thereby allow the patient's external abdominal oblique muscles to contract to generate a forceful airway clearing coughs. In addition, the reflex closing of the larynx during swallowing helps protect the patient from aspirating food or other foreign material into the respiratory airways. Medical conditions which bring about impairment of the laryngeal cough reflex include operative anesthesia, neurological deficits such as seen in strokes, neuromuscular disease, extubation, drug-induced laryngeal suppression, and others.

The patient's ability to produce an involuntary cough, and the strength of that cough, provide measures of the status of the laryngeal cough reflex. The inventors have previously described the use of compositions of L-tartaric acid for stimulating sensory innervations associated with the patient's larynx to thereby induce a forceful involuntary cough.

Previously, however, evaluating the strength of a patient's induced cough was dependent on personal observation by a skilled physician, and was consequently a qualitative and somewhat subjective measure the functional status of the laryngeal cough reflex.

Accordingly, there has been a need for a quantitative technique for determining whether a patient's laryngeal cough reflex has been impaired, and/or fully restored, and thus indicative of whether the patient remains susceptible to an aspiration event due to an impaired laryngeal cough reflex.

The present invention discloses a nebulizer having a trigger for activating an electromyogram (EMG) machine, and a method of quantitating the patient's involuntary cough reflex in response to nebulized administration of a cough-inducing substance to the patient's throat and/or larynx.

### SUMMARY OF THE INVENTION

With the foregoing in mind, the present invention advantageously includes an apparatus which comprises a nebulizer containing a composition of L-tartaric acid, a switch associated with the nebulizer and responsive to actuation of the nebulizer, a connection between the switch and an EMG machine for triggering the machine to begin recording responsive to the switch, and the appropriate EMG electrodes for monitoring electrical impulses produced by a patient's external abdominal oblique muscles, which are the muscles primarily involved in generating a cough.

The nebulizer, as noted above, preferably contains a composition made with L-tartaric acid, and is disposed with a switch which is activated to close an electrical circuit when a patient inhales the composition through the nebulizer. The switch is connected to the EMG machine, so that when the switch closes the circuit, the EMG machine is activated to start recording. In a patient with a fully functional laryngeal cough reflex, the L-tartaric acid composition induces glottal closure in the larynx, followed by a strong contraction of the external abdominal oblique muscles to produce a forceful involuntary cough.

By measuring the time lapse, also known as latency, from inhalation/activation of the nebulizer to the arrival of the cough-producing electrical stimulus at the external abdominal oblique muscles, we obtain a quantitative measure of the strength of the cough reflex. Once the normal range for this latency is established, we can quantitatively evaluate a patient for laryngeal cough reflex functionality by comparing the patient's measured latency with the expected normal range. A complete absence of laryngeal function results in infinite latency, that is, no cough is produced at all. As the patient recovers a functional laryngeal cough reflex, the latency period decreases until full functionality is achieved, at which time the latency is within a normal range.

An apparatus according to the present invention will preferably comprise a nebulizer which incorporates a switch as part of the nebulizer. Accordingly, the invention includes the described apparatus and a method for quantitatively testing and evaluating laryngeal cough reflex function, as described above. The apparatus and associated method should be applicable in many medical situations involving impairment and/or recovery of the laryngeal cough reflex.

### BRIEF DESCRIPTION OF THE DRAWINGS

Some of the features, advantages, and benefits of the present invention having been stated, others will become apparent as the description proceeds when taken in conjunction with the accompanying drawings, which are presented solely for exemplary purposes and not with intent to limit the invention thereto, and in which:

FIG. 1 is a diagrammatic view of an apparatus according to an embodiment of the present invention, showing patient using an inhalation activated nebulizer having a switch which is connected by a wire to an EMG machine;

FIG. 2 is a block diagram showing the method of the present invention; and

FIG. 3 shows the invention of FIG. 1 in use with patients.

### DETAILED DESCRIPTION OF THE PREFERRED EMBODIMENT

The present invention will now be described more fully hereinafter with reference to the accompanying drawings, in which preferred embodiments of the invention are shown. Unless otherwise defined, technical and scientific terms used herein have the same meaning as commonly understood by one of ordinary skill in the art to which this invention pertains. Although methods and materials similar or equivalent to those described herein can be used in the practice or testing of the present invention, suitable methods and materials are described below. Any publications, patent applications, patents, and other references mentioned herein are incorporated by reference in their entirety. In case of conflict, the present specification, including any definitions, will control. In addition, the materials, methods and examples given are illustrative in nature only and not intended to be limiting. Accordingly, this invention may be embodied in many different forms and should not be construed as limited to the illustrated embodiments set forth herein. Rather, these illustrated embodiments are provided solely for exemplary purposes so that this disclosure will be thorough and complete, and will fully convey the scope of the invention to those skilled in the art. Other features and advantages of the invention will become apparent from the following detailed description, and from the claims.

At the outset, the skilled should understand that the term “nebulizer” as used herein refers to any device for atomizing a substance. The process of atomizing is generally recognized to be the mechanical subdivision of a bulk liquid into droplets, although solid materials may also be atomized. The droplets produced, however, may be in various average size ranges and the resulting atomized liquid is usually described by different terms according to general size of the droplets produced. For example, while the terminology is not standardized in the art, the term “spraying” is generally taken to indicate the production of coarse drops usually in the range of about 100-1000 μm in average diameter. Similarly, the term “sprinkling” generally indicates droplets of an even coarser nature and usually in the range of about greater than 1000 μm in average diameter. Also, the term “misting” is often employed to designate production of fine droplets in the size range of about 10-100 μm in average diameter, and the term “nebulizing” typically indicates production of very fine droplets in the size range of about less than 10 μm in average diameter. It is also known that particles, and droplets, having an average aerodynamic diameter of less than about 10 μm are more likely to travel into the smallest reaches of the respiratory airways, the alveoli, so that nebulizing is often used to introduce droplets into the respiratory system. Notwithstanding the above noted terms indicating various size ranges for the atomized liquid, it should be understood that while a preferred embodiment of the present invention is described herein using the term “nebulizer”, the invention is intended to include any atomization device and process, including liquids and solids, and that, preferably, the invention includes a nebulizer typical of the various types used for medical treatment purposes. Additionally, the nebulizer of the present invention may operate using any nebulizer geometry and any type of motivating force for generating the atomized fluid, for example, hydraulic, pneumatic, vibrational, rotary, electrostatic, ultrasonic, and others. The nebulizer may also be actuated simply by inhaling, that is, taking a breath through the device. One suitable nebulizer structure for use in the invention is, for example, that described in U.S. Pat. No. 6,044,841, although many others may be used as well.

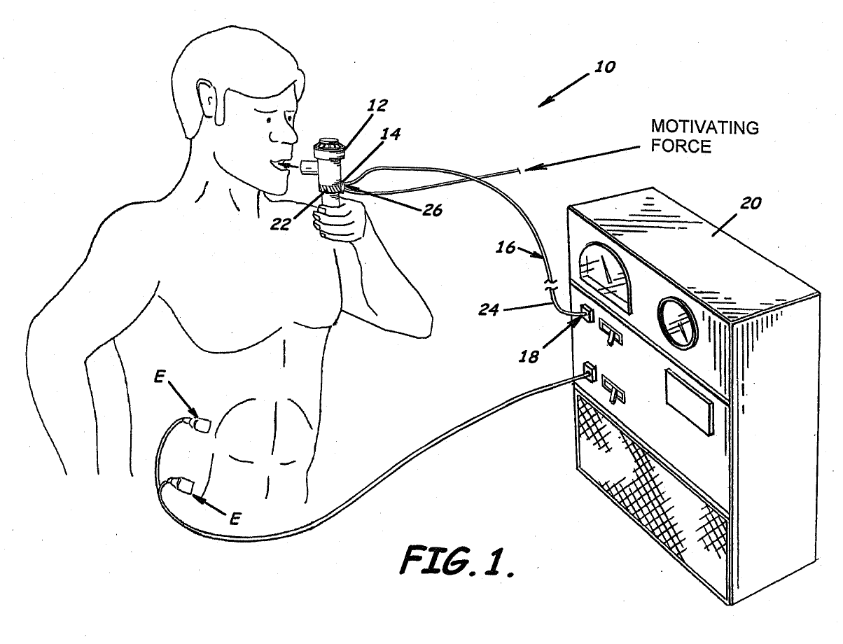

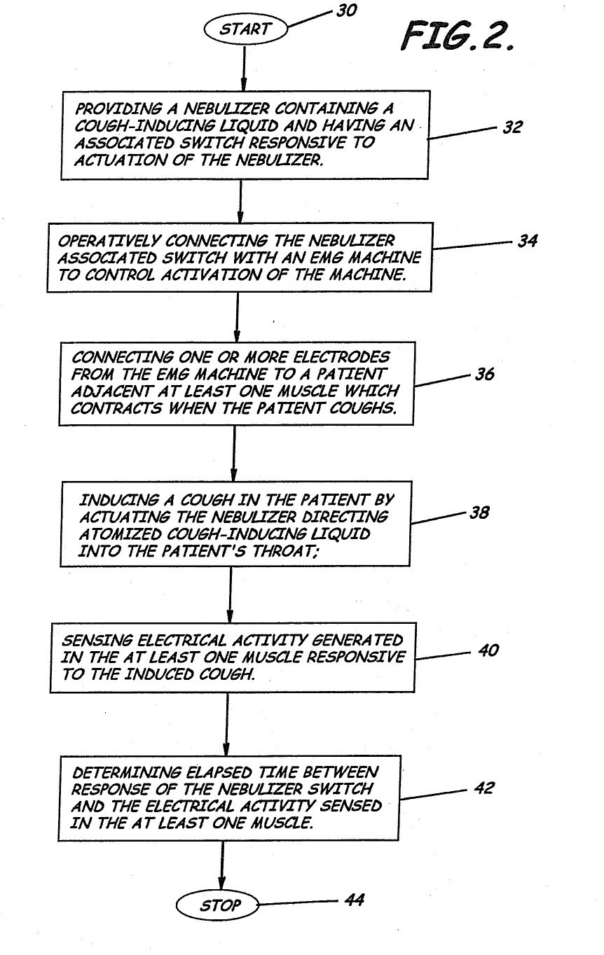

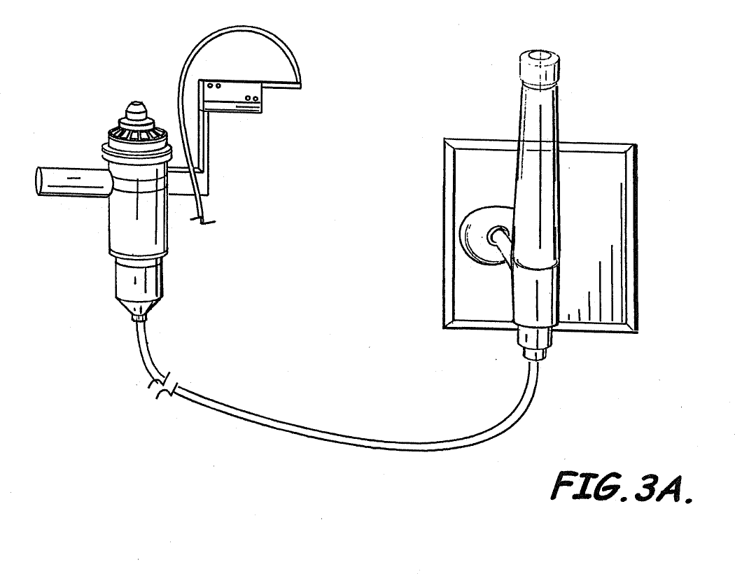

FIGS. 1 and 2 illustrate an apparatus 10 for evaluating a patient's laryngeal cough reflex function according to the present invention, and its associated method. The apparatus comprises a nebulizer 12, a switch 14 associated with the nebulizer, and a connection 16 extending from the switch to an end 18 operably connectable to an EMG machine 20. The nebulizer is capable of being actuated to atomize a cough-inducing substance 22 contained therein. Further, the nebulizer may preferably be inhalation actuated or manually actuated to atomize the cough-inducing substance 22.

The switch 14 associated with the nebulizer 12 is responsive to actuation of the nebulizer, so that when the nebulizer is actuated, the switch is triggered, typically to the ON position. The switch 14 may be a microswitch such as one manufactured by Cherry Electric as part #e22851-0, however, the skilled will recognize that any one of a large variety of switches may be adapted for use in the present invention.

A connection 16 extends between the switch 14 and an EMG machine 20 to thereby activate the EMG machine responsive to the triggering of the switch. Accordingly, when a patient takes a breath through the nebulizer 12, the nebulizer associated switch 14 is triggered and a signal travels via the connection 16 to the EMG machine 20 to thereby activate the machine. In the apparatus 10, connection 16 between the switch 14 and the EMG machine 20 may comprise at least one wire 24 having a first end 26 connected to the electrical switch and having a second end 18 connectable to n EMG machine 20. It is to be understood that EMG machine 20 is best equipped or adapted with a port for therein receiving the connection 16 to the nebulizer associated switch 14. Alternatively, in the apparatus 10 the described connection 16 between the switch 14 and EMG machine 20 may be a wireless connection, and could rely on light, especially an infrared light signal, such as employed in a typical television remote control unit or remote mouse device for a personal computer. In this alternative embodiment, EMG machine 20 is preferably equipped with or adapted to include a sensor which detects and responds to the light signal from the nebulizer associated switch 14.

In the present apparatus 10, the cough-inducing substance 22 may contain one or more salts of tartaric acid and, preferably, the cough-inducing substance is made with up to approximately 20% tartaric acid, which is harmless irritant and an effective cough-inducing substance.

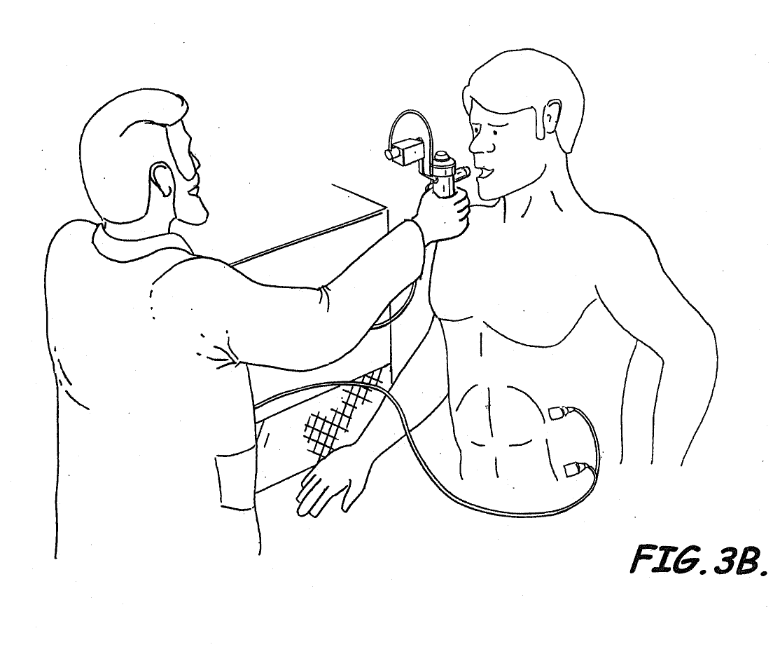

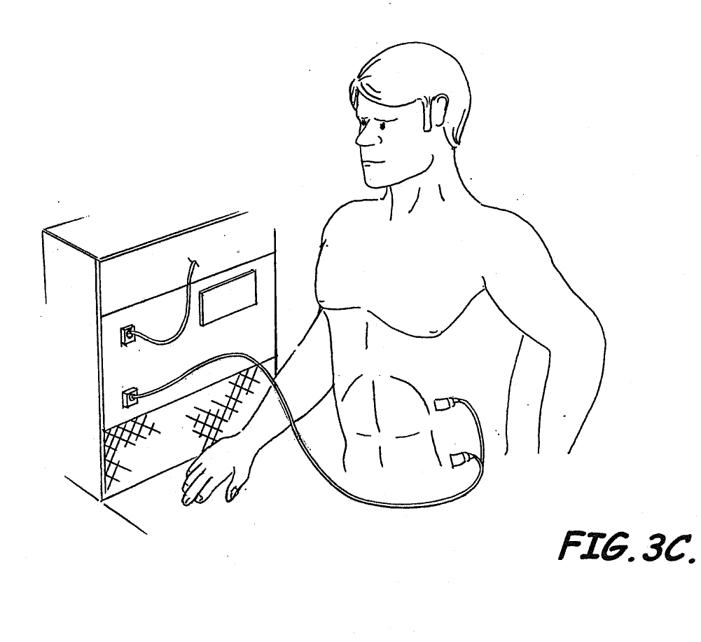

A further aspect of the invention includes an apparatus 10 for evaluating a patient's laryngeal cough reflex function, the apparatus comprising in combination a nebulizer 12, an electrical switch 14 responsive to actuation of the nebulizer, an EMG machine 20, and a connection 16 between the switch and the EMG machine. This apparatus 10 combination includes all the features described above and, in addition, includes EMG machine 20 as part of the inventive apparatus. As known to the skilled, an EMG machine 20 is used for sensing muscular electrical activity in a patient being tested, as shown in FIG. 3, wherein FIG. 3A is a view of the nebulizer device having an associated switch mechanism, FIG. 3B shows a technician preparing to nebulize a patient, and FIG. 3C shows electrodes positioned on a patient for measuring electrical activity of external abdominal oblique muscles. A method aspect of the invention is illustrated in FIG. 2 and from the start 30 includes evaluating a patient's laryngeal cough reflex function by use of the described apparatus 10. The method comprises first providing 32 a nebulizer containing a cough-inducing substance 22, the nebulizer being associated with a switch 14 responsive to actuation of the nebulizer. The method continues by operatively connecting 34 the nebulizer associated switch 14 with an EMG machine 20 so as to activate the EMG machine responsive to the nebulizer switch. The method also calls for connecting 36 one or more sensing electrodes E from EMG machine 20 to the patient at a position sufficiently close to at least one muscle which contracts when the patient coughs, so as to sense electrical activity in the at least one muscle. Typically electrodes are adhered to the patient's skin in an area overlying the muscle of interest. A cough is then induced 38 in the patient by actuating the nebulizer 12 so as to direct atomized cough-inducing substance 22 into the patient's throat. Electrical activity generated in the at least one muscle responsive to the induced cough is then sensed 40 through the one or more electrodes connected from the patient to EMG machine 20. Finally, the method includes determining elapsed time 42 between response of the nebulizer switch 14 and the electrical activity sensed in the at least one muscle. Thereafter the method stops at 44.

Other aspects of the method include wherein the cough-inducing substance 22 preferably contains one or more salts of tartaric acid and wherein the substance is best made with up to approximately 20% tartaric acid. Additionally, in the method the at least one muscle of the patient preferably consists of an external abdominal oblique muscle. Also, in the method inducing a cough most preferably comprises contacting the patient's larynx with the atomized cough-inducing substance 22.

Quantitative aspects of the method include an elapsed time measured from actuation of the switch 14 to initiation of muscle electrical activity of approximately from 15 to 21 milliseconds, which indicates a normal laryngeal cough reflex. In the method, an elapsed time of greater than approximately 21 milliseconds from actuation of the switch 14 to sensing electrical activity in the at least one muscle indicates an impaired laryngeal cough reflex. Those skilled in the art will readily understand that the degree of impairment of the patient's laryngeal cough reflex is relatively proportional to the elapsed time between switch 14 actuation and initiation of muscle electrical activity. Beyond the normal range, the longer the elapsed time, the more severe the impairment of the laryngeal cough reflex. Of course, complete impairment of the reflex will result in no cough production at all, so that elapsed time approaches infinity.

In the drawings and specification, there have been disclosed a typical preferred embodiment of the invention, and although specific terms are employed, the terms are used in a descriptive sense only and not for purposes of limitation. The invention has been described in considerable detail with specific reference to these illustrated embodiments. It will be apparent, however, that various modifications and changes can be made within the spirit and scope of the invention as described in the foregoing specification and as defined in the appended claims.

1. An apparatus for evaluating a patient's laryngeal cough reflex function, said apparatus comprising:
a nebulizer capable of being actuated to atomize a cough-inducing substance.
a switch associated with said nebulizer, said switch responsive to actuation of the nebulizer; and
a connection between said switch and an EMG machine to thereby transmit a signal to activate the EMG machine responsive to said switch.

2. The apparatus of claim 1, wherein said connection comprises at least one wire having a first end connected to said electrical switch and having a 15 second end connectable to an EMG machine.

3. The apparatus of claim 1, wherein said connection is a wireless connection.

4. The apparatus of claim 1, wherein said connection comprises light to carry a signal.

5. The apparatus of claim 1, wherein said connection comprises infrared light to carry a signal.

6. The apparatus of claim 1, wherein the cough-inducing substance contains one or, more salts of tartaric acid.

7. The apparatus of claim 1, wherein the cough-inducing substance is made with up to approximately 20% tartaric acid.

8. The apparatus of claim 1, wherein the nebulizer is inhalation actuated to atomize the cough-inducing substance.

9. (canceled)

10. (canceled)

11. The apparatus of claim 9, wherein said connection is a wireless connection.

12. The apparatus of claim 9, wherein said connection comprises light to carry a signal.

13. The apparatus of claim 9, wherein said connection comprises infrared light to carry a signal.

14. (canceled)

15. (canceled)

16. (canceled)

17. A method of evaluating a patient's laryngeal cough reflex function, the method comprising:
providing a nebulizer containing a cough-inducing substance, 10 the nebulizer being associated with a switch responsive to actuation of the nebulizer;
operatively connecting the nebulizer associated switch with an EMG machine so as to activate the EMG machine responsive to the switch;
connecting one or more sensing electrodes from the EMG machine to a position on the patient sufficiently close to at least one muscle which contracts to cause the patient to cough, so as to sense electrical, activity in the at least one muscle;
inducing a cough in the patient by actuating the nebulizer so as 20 to direct atomized cough-inducing substance into the patient's throat;
sensing electrical activity generated in the at least one muscle responsive to the induced cough; and
determining elapsed time between response of the nebulizer switch and the electrical activity sensed in the at least one muscle.

18. The method of claim 17, wherein the cough-inducing substance contains one or more salts of tartaric acid.

19. The method of claim 17, wherein the cough-inducing substance is made with up to approximately 20% tartaric acid.

20. The method of claim 17 wherein the at least one muscle of the patient consists of an external abdominal oblique muscle.

21. The method of claim 17, wherein inducing a cough further comprises contacting the patient's larynx with the atomized cough-inducing substance.

22. The method of claim 17, wherein an elapsed time in a range of between approximately 15 to 21 milliseconds indicates a normal laryngeal cough reflex.

23. The method of claim 17, wherein an elapsed time in a range of greater than approximately 21 milliseconds indicates an impaired laryngeal cough reflex.
